(19) United States
(12) Patent Application Publication
(10) Pub. No.: US 2006/0127886 A1
(43) Pub. Date: Jun. 15, 2006

(54) Sample-efficient lateral flow immunoassay

(76) Inventors: Rosann Marie Kaylor, Cumming, GA (US); Ning Wei, Roswell, GA (US); Chibueze Chidebelu-Eze, Atlanta, GA (US); Shawn Ray Feaster, Duluth, GA (US); Paul Christopher, Pontypridd (GB)

Correspondence Address: KIMBERLY-CLARK WORLDWIDE, INC., 401 NORTH LAKE STREET, NEENAH, WI, 54956, US

(21) Appl. No.: 11/012,759

(22) Filed: Dec. 15, 2004

(51) Int. Cl. C12Q 1/70; G01N 33/554; G01N 33/53; G01N 33/558; G01N 33/569; C12Q 1/68

(52) U.S. Cl. 435/5; 436/514; 435/7.32; 435/7.5; 435/6; 435/7.22; 435/7.31

(57) ABSTRACT

There is provided a lateral flow assay device for detecting the presence or quantity of an analyte residing in a test sample where the lateral flow assay device has a porous membrane in communication with a conjugate pad and a wicking pad. The porous membrane has a detection zone which has an immobilized first capture reagent configured to bind to at least a portion of the analyte and analyte-conjugate complexes to generate a detection signal. A control zone may be located downstream from the detection zone on the porous membrane and has a second capture reagent immobilized within the control zone. The conjugate pad is located upstream from the detection zone, and has detection probes with specific binding members for the analyte. The sample is deposited between the control and detection zones. A buffer release zone is located upstream of the conjugate pad and provides for buffer addition to the device, the buffer serving to move the detection probes to the detection and control zones.

### BACKGROUND OF THE INVENTION

There are several well-known immunoassay methods that use immunoreactants labeled with a detectable component so that the analyte may be detected analytically. For example, “sandwich-type” assays typically involve mixing the test sample with detectable probes, such as dyed latex or a radioisotope, which are conjugated with a specific binding member for the analyte. The conjugated probes form complexes with the analyte. These complexes then reach a zone of immobilized antibodies where binding occurs between the antibodies and the analyte to form ternary “sandwich complexes.” The sandwich complexes are localized at the zone for detection of the analyte. This technique may be used to obtain quantitative or semi-quantitative results. An alternative technique is the “competitive-type” assay. In a “competitive-type” assay, the label is typically a labeled analyte or analyte-analogue that competes for binding of an antibody with any unlabeled analyte present in the sample. Competitive assays are typically used for detection of analytes such as haptens, each hapten being monovalent and capable of binding only one antibody molecule.

Another type of assay is the inhibition/overflow assay where an analyte is striped on a first line, and an antibody that is specific to the antibody on the conjugate particles (e.g., goat anti-mouse or “GAM”) is striped next. In this assay, any analyte that is present will bind to the conjugate particles having an antibody (or other binder) that is specific to the analyte. At levels below a certain threshold amount of analyte, the particles will not be fully covered, i.e. not fully inhibited, by the analyte, and hence will still be able to form a complex at the first line striped with analyte. This inhibition line essentially acts to remove/bind conjugate when the analyte is below a threshold level. The next line, which consists of an antibody that recognizes the antibody on the conjugate particles, would act as an “overflow” line. It binds particles (thereby causing a colored line to form) only in the case of analyte levels above the threshold level. In addition, a positive control line can be used, such as a goat anti-rabbit (“GAR”) line striped to capture a different population of conjugate particles (e.g., those having rabbit antibody on them, for example). This line should always form as long as the test is viable and has been performed properly.

Flow through or lateral-flow assays have become more common for many analytes but many require a relatively large sample size in order to allow for the flow of the sample and label or conjugate particles that is characteristic of the lateral flow assay. More particularly, currently available lateral flow assays employ conjugates that are located downstream from a sample deposition point and upstream from a detection zone. The sample itself is relied upon to re-suspend the conjugates and carry them to the detection zone. Alternatively, additional diluent can be added with the sample to carry the sample to the conjugate pad, aid in re-suspending the conjugate particles, and then reach the detection zone. In either case, the sample addition is typically applied upstream from the conjugate particles to aid in the re-suspension of the particles. Note that “upstream” and “downstream” refer to the position of an item relative to the direction of flow of a sample on the assay device.

Despite the benefits achieved from these devices, they do not always produce the desired signal (line) intensity. This limits the circumstances in which they may be used, since the signal may not be visible at low levels of analyte. Conventional assays may also be rendered less reliable because of the intense red color of a (blood) sample or because of the viscosity of the sample which may cause problems with sample flow. A need exists, therefore, for an improved technique of assaying that can give stronger signal (line) intensities, as well as reliable results without requiring a large sample volume.

### SUMMARY OF THE INVENTION

In accordance with one embodiment of the present invention, an assay device for detecting the presence or quantity of an analyte residing in a test sample is disclosed. The assay device comprises a conjugate pad that is in liquid communication with a porous membrane that is also in communication with a wicking pad. It should be noted that it is possible that the conjugate pad, porous membrane and wicking pad may be a single material having the functionality of all three areas.

The porous membrane may be made from any of a variety of materials through which the detection probes are capable of passing like, for example, nitrocellulose. The porous membrane has a detection zone where the first capture reagent is immobilized. The first capture reagent is configured to bind to at least a portion of the analyte and analyte-conjugate complexes to generate a detection signal. The first capture reagent may be selected from the group consisting of antigens, haptens, protein A or G, neutravidin, avidin, streptavidin, captavidin, primary or secondary antibodies, and complexes thereof. The first capture reagent may, for example, bind to complexes formed between the analyte and the conjugated detection probes.

The control zone is located on the porous membrane downstream from the detection zone. A second capture reagent is immobilized within the control zone that is configured to bind to the conjugate, conjugate-analyte complex or pure probes, to indicate the assay is performing properly. In one embodiment, the second capture reagent is selected from the group consisting of antigens, haptens, polyelectrolytes, protein A or G, neutravidin, avidin, streptavidin, captavidin, primary or secondary antibodies, and complexes thereof.

The conjugate pad contains detection probes that signal the presence of the analyte. The conjugate pad may also include other, different probe populations, including probes for indication at the control zone. If desired, the detection probes may comprise a substance selected from the group consisting of chromogens, catalysts, luminescent compounds (e.g., fluorescent, phosphorescent, etc.), radioactive compounds, visual labels, particles (e.g., dyed, gold, silver, other optically-dense materials), liposomes, and combinations thereof. The specific binding member may be selected from the group consisting of antigens, haptens, aptamers, primary or secondary antibodies, biotin, and combinations thereof.

In liquid communication with the end of the conjugate pad away from the membrane there is a buffer release zone. After the sample has been deposited on the device between the conjugate and detection zones, a buffer is released from upstream in the buffer release zone. The buffer washes probes from the conjugate pad toward the detection zone where the probes will be captured on the detection zone by the analyte, if present, and yield a positive result. If the sample contains no analyte, the detection line will be negative. The buffer mixture, still containing some probes (which may include probes different from the detection probes), continues to the control zone where a reagent captures conjugate, conjugate-analyte complex or pure probes to indicate the assay is functioning properly.

The wicking pad is in liquid communication with the membrane and provides a driving force for liquid movement.

The method involves adding the sample downstream from the particles, rather than in the conventional location which is upstream from the particles. After deposition of the sample, the diluent is released, at a point on the test strip upstream from the particles. The diluent then provides the required fluid to re-suspend the particles so that they can flow down the test strip. After contacting the particles, the diluent-particle mixture flows down to the point of contacting the sample (e.g., blood) for the remainder of the assay. It has been found that this method increases the line signal intensity in some cases.

In accordance with another embodiment of the present invention, a method for detecting the presence or quantity of an analyte residing in a test sample is disclosed. The method includes the steps of

i) providing a lateral flow assay device having a porous membrane in liquid communication with a conjugate pad and a wicking pad, the conjugate pad having detection probes conjugated with a specific binding member for the analyte, the porous membrane defining a detection zone in which a first capture reagent is immobilized and a control zone within which a second capture reagent is immobilized, wherein the control zone is located downstream from the detection zone, the conjugate pad is located upstream of the porous membrane and the buffer release zone is upstream of the conjugate pad;

ii) contacting the test sample containing the analyte with the device downstream from the conjugate pad;

iii) releasing a buffer at the buffer release zone so that the buffer will carry the detection probes to the sample application area, then to the detection and control zones;

iv) detecting the detection signal.

Other features and aspects of the present invention are discussed in greater detail below.

### BRIEF DESCRIPTION OF THE DRAWINGS

FIG. 1 is a perspective view of one embodiment of a lateral flow assay device of the present invention.

### DETAILED DESCRIPTION

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, yeasts, fungi, protozoa, and metabolites of or antibodies to any of the above substances. Specific examples of some analytes include ferritin; creatinine kinase MB (CK-MB); digoxin; phenyloin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651.

As used herein, the term “test sample” generally refers to a material suspected of containing the analyte. The test sample may, for instance, include materials obtained directly from a source, as well as materials pretreated using techniques, such as, but not limited to, filtration, precipitation, dilution, distillation, mixing, concentration, inactivation of interfering components, the addition of reagents, lysing, and so forth. The test sample may be derived from a biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, amniotic fluid or the like. Besides physiological fluids, other liquid samples may be used, such as water, food products, and so forth. In addition, a solid material suspected of containing the analyte may also be used as the test sample.

In general, the present invention is directed to a lateral flow assay device for detecting the presence or quantity of an analyte residing in a test sample.

In conventional lateral flow methods, the sample is typically applied upstream from the location of the immobilized conjugate particles, such that the sample can help re-suspend the particles to allow the test to proceed. In contrast, however, the instant invention discloses a novel method and/or location to apply the sample in order to reduce or eliminate problems associated with low-volume samples, such as, for example, in blood-based assays. In the case of specialized applications where the sample volume may be more limited (e.g., blood-based and swab-based applications), a diluent can be used to mix with the sample to thereby decrease the amount of body fluid required. In these cases, the diluent itself can cause re-suspension of the immobilized particles.

Known assays require that the targeted analyte in a sample moves from a point of deposition to a point where it may be detected. Known assays move the sample through an area containing conjugate particles and then to a detection zone. Some of these known assays use a liquid diluent or “buffer” to move the sample to the conjugate particles and on to the detection zone.

In contrast to the known assays, the instant device allows the sample to the deposited downstream from the conjugate particles. A diluent (e.g., buffer) is thereafter released and moves the particles, initially located on a conjugate pad, to the sample located between the particles and a detection zone.

The inventors have discovered that allowing the detection particles to move with the diluent to the sample which has been added downstream of the particles, enables the use of samples of a very small volume. The diluent serves to very efficiently re-suspend the detection probes so that they may move farther down the conjugate pad and along the membrane to provide a test result. This method is counter-intuitive since the liquid sample in conventional lateral flow devices is used to re-suspend the conjugate particles. In addition, it is conventionally desired that the sample and particles are in contact in order for the immunoassay and binding to occur. It has been surprisingly found, however, that the instant method wherein the sample is applied downstream from the particles, and then “chased” by the re-suspended conjugate particles in the diluent, may actually increase the signal intensity in some cases.

The device utilizes a porous membrane having a conjugate pad, a sample application zone and a detection zone. The detection zone has immobilized capture reagents. The sample application zone may be a location on the conjugate pad material that is downstream from the immobilized particles, or it may be a front location on the membrane (upstream of the detection zone), or it may be a separate material that is located between the conjugate pad and the membrane with the detection zone. The device further uses a diluent release zone on the upstream end of the device before the sample application zone and a conjugate pad located between the diluent release zone and the sample. A wicking pad is in liquid communication with the opposite end of the porous membrane on the downstream end of the device. In use, the sample is applied in the sample application zone and after a period of time, the diluent is released. The diluent re-suspends and carries the conjugate particles to the sample and still farther downstream to the detection zone, resulting in an indication of the presence of analyte.

Examples of suitable analytes that may be detected using the invention include, but are not limited to toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, yeasts, fungi, protozoa, and metabolites of or antibodies to any of the above substances. Specific examples of some analytes include ferritin; creatinine kinase MB (CK-MB); digoxin; phenyloin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651.

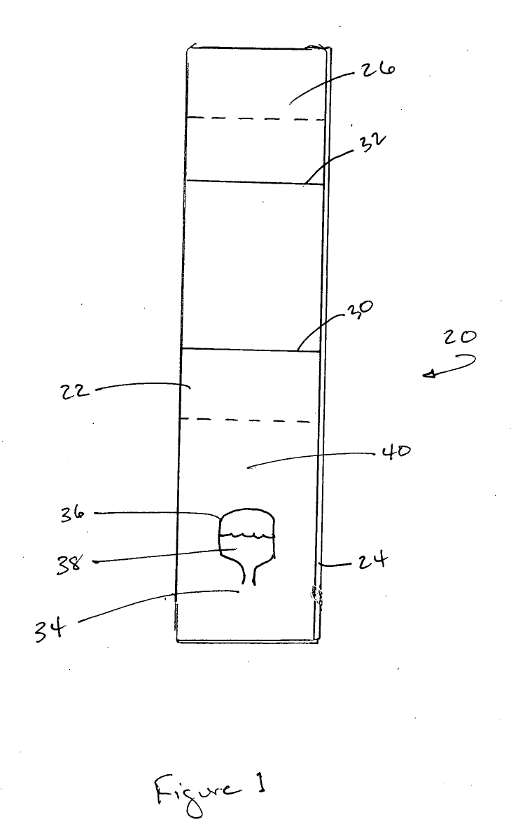

Referring to FIG. 1, one embodiment of a lateral flow assay device 20 that may be formed will be described in more detail. It should be noted that the term “lateral flow” is meant to be descriptive and not limiting, as the device could be configured in other ways with the same effect. Radial or vertical flow devices can easily be envisioned, for example, employing the same principle as the instant invention, without departure from the spirit of the invention. As shown, the device 20 contains a porous membrane 22 optionally supported by a rigid material 24. The porous membrane 22 has a detection zone (or line) 30. The porous membrane 22 may also have a control zone (or line) 32.

In general, the porous membrane 22 may be made from any of a variety of materials through which the detection probes are capable of passing. For example, the materials used to form the porous membrane 22 may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and the like. In one particular embodiment, the porous membrane 22 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms. Suitable membranes include nitrocellulose membranes HF075 and HF120 from Millipore Corporation of Billerica, Mass., USA.

The device 20 may also contain a wicking pad 26. The wicking pad 26 generally receives fluid that has migrated through the entire porous membrane 22. As is well known in the art, the wicking pad 26 may assist in promoting capillary action and fluid flow through the membrane 22.

The device 20 has a diluent release zone 34. In one embodiment the diluent release zone 34 has a diluent reservoir 36 within which may be stored the diluent 38. Diluent 38 may alternatively be supplied by a separate reservoir. The diluent 28 may be any liquid that will carry away the detection probes used in the invention. Examples of suitable diluentsinclude phosphate buffered saline (PBS) solution (pH of 7.2), tris-buffered saline (TBS) solution (pH of 8.2) or 2-(N-morpholino) ethane sulfonic acid (MES) (pH of 5.3). These may contain other additives to aid the performance of the assay, such as surfactants, water-soluble polymers, proteins, blockers to prevent non-specific binding, and preservatives.

A conjugate pad 40 is in liquid communication with the diluent release zone 34 and is located between the diluent release zone 34 and the porous membrane 22 so that as the diluent 38 moves from the diluent release zone 34 it will traverse the conjugate pad 40 and carry conjugate particles to the detection zone 30 and the control zone 32 on the porous membrane 22. The conjugate pad 40 is formed from a material through which the diluent is capable of passing. The conjugate pad 40 may be formed from glass fibers, for example. Although only one conjugate pad 40 is shown, it should be understood that other conjugate pads may also be used in the present invention.

To initiate the detection of an analyte within the test sample, a user may directly apply, contact or deposit the test sample to an application zone 42 between the conjugate pad 40 and detection zone 30 portion of the porous membrane 22. Once a sample has contacted the application zone 42, diluent 38 is released into the diluent release zone 34. The diluent 38 may be applied by means of an integral reservoir, or by a separate source such as by pipette or any other effective means known to those skilled in the art. The diluent 38 travels through the conjugate pad 40 that is in liquid communication with the porous membrane 22, to the application zone 42, the detection zone 30 and the control zone 32.

A predetermined amount of at least one type of conjugate particles is applied on the conjugate pad in order to facilitate accurate detection of the presence or absence of an analyte within the test sample. Any substance generally capable of generating a signal that is detectable visually or by an instrumental device may be used as detection probes. Various suitable substances may include chromogens; catalysts; luminescent compounds (e.g., fluorescent, phosphorescent, etc.); radioactive compounds; visual labels, including colloidal metallic (e.g., gold) and non-metallic particles, dyed particles, enzymes or substrates, or organic polymer latex particles; liposomes or other vesicles containing signal producing substances; and so forth. Some enzymes suitable for use as detection probes are disclosed in U.S. Pat. No. 4,275,149. One example of an enzyme/substrate system is the enzyme alkaline phosphatase and the substrate nitro blue tetrazolium-5-bromo-4-chloro-3-indolyl phosphate, or derivative or analog thereof, or the substrate 4-methylumbelliferyl-phosphate. Other suitable conjugate particles may be described in U.S. Pat. Nos. 5,670,381 and 5,252,459. In some embodiments, the conjugate particles may contain a fluorescent compound that produces a detectable signal. The fluorescent compound may be a fluorescent molecule, polymer, dendrimer, particle, and so forth. Some examples of suitable fluorescent molecules, for instance, include, but are not limited to, fluorescein, europium chelates, phycobiliprotein, rhodamine and their derivatives and analogs.

The conjugate particles, such as described above, may be used alone or in conjunction with a microparticle (sometimes referred to as “beads” or “microbeads”). For instance, naturally occurring microparticles, such as nuclei, mycoplasma, plasmids, plastids, mammalian cells (e.g., erythrocyte ghosts), unicellular microorganisms (e.g., bacteria), polysaccharides (e.g., agarose), and so forth, may be used. Further, synthetic microparticles may also be utilized. For example, in one embodiment, latex microparticles that are labeled with a fluorescent or colored dye are utilized. Although any latex microparticle may be used in the present invention, the latex microparticles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable microparticles may be described in U.S. Pat. Nos. 5,670,381 and 5,252,459. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 1 nanometer to about 100 microns, and in some embodiments, from about 10 nanometers to about 10 microns. For instance, “micron-scale” particles are often desired. When utilized, such “micron-scale” particles may have an average size of from about 1 micron to about 1,000 microns, in some embodiments from about 1 micron to about 100 microns, and in some embodiments, from about 1 micron to about 10 microns. Likewise, “nano-scale” particles may also be utilized. Such “nano-scale” particles may have an average size of from about 0.1 to about 80 nanometers, in some embodiments from about 0.1 to about 5 nanometers, and in some embodiments, from about 1 to about 20 nanometers.

In some instances, it is desired to modify the particles in some manner so that they are more readily able to bind to the analyte. In such instances, the particles may be modified with certain specific binding members that are adhered thereto to form conjugated particles. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The specific binding members may generally be attached to the particles using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the particle may contain a relatively high surface concentration of polar groups. In addition, although conjugate particles are often functionalized after synthesis, in certain cases, such as poly(thiophenol), the microparticles are capable of direct covalent linking with a protein without the need for further modification.

Referring again to FIG. 1, the assay device 20 contains a detection zone 30 within which is immobilized a first capture reagent that is capable of binding to the analyte or to the conjugate particle-analyte complex. The binding of the analyte results in a detectible indication that the analyte is present and such an indication may be visual or through other means such as various detectors or readers (e.g., fluorescence readers), discussed below. Readers may also be designed to determine the relative amounts of analyte at the detection site, based upon the intensity of the signal at the detection zone.

In some embodiments, the first capture reagent may be a biological capture reagent. Such biological capture reagents are well known in the art and may include, but are not limited to, antigens, haptens, protein A or G, neutravidin, avidin, streptavidin, captavidin, primary or secondary antibodies (e.g., polyclonal, monoclonal, etc.), and complexes thereof. In many cases, it is desired that these biological capture reagents are capable of binding to a specific binding member (e.g., antibody) present on the conjugate particles.

It may also be desired to utilize various non-biological materials for the capture reagent. For instance, in some embodiments, the reagent may include a polyelectrolyte. The polyelectrolytes may have a net positive charge or a negative charge, or a net charge that is generally neutral. Some suitable examples of polyelectrolytes having a net positive charge include, but are not limited to, polylysine (commercially available from Sigma-Aldrich Chemical Co., Inc. of St. Louis, Mo.), polyethylenimine; epichlorohydrin-functionalized polyamines and/or polyamidoamines, such as poly(dimethylamine-co-epichlorohydrin); polydiallyldimethyl-ammonium chloride; cationic cellulose derivatives, such as cellulose copolymers or cellulose derivatives grafted with a quaternary ammonium water-soluble monomer; and so forth. In one particular embodiment, CelQuat® SC-230M or H-100 (available from National Starch & Chemical, Inc.), which are cellulosic derivatives containing a quaternary ammonium water-soluble monomer, may be utilized. Some suitable examples of polyelectrolytes having a net negative charge include, but are not limited to, polyacrylic acids, such as poly(ethylene-co-methacrylic acid, sodium salt), and so forth. It should also be understood that other polyelectrolytes may also be used. Some of these, such as amphiphilic polyelectrolytes (i.e., having polar and non-polar portions) may have a net charge that is generally neutral. For instance, some examples of suitable amphiphilic polyelectrolytes include, but are not limited to, poly(styryl-b-N-methyl 2-vinyl pyridinium iodide) and poly(styryl-b-acrylic acid), both of which are available from Polymer Source, Inc. of Dorval, Canada.

The first capture reagent serves as a stationary binding site for complexes formed between the analyte and the conjugate particles. Specifically, analytes, such as antibodies, antigens, etc., typically have two or more binding sites (e.g., epitopes). Upon reaching the detection zone 30, one of these binding sites is occupied by the specific binding member of the probe. However, the free binding site of the analyte may bind to the immobilized capture reagent. Upon being bound to the immobilized capture reagent, the complexed probes form a new ternary sandwich complex.

The detection zone 30 may generally provide any number of distinct detection regions so that a user may better determine the concentration of a particular analyte within a test sample. Each region may contain the same capture reagents, or may contain different capture reagents for capturing multiple analytes. For example, the detection zone 30 may include two or more distinct detection regions (e.g., lines, dots, etc.). The detection regions may be disposed in the form of lines in a direction that is substantially perpendicular to the flow of the test sample through the assay device 20. Likewise, in some embodiments, the detection regions may be disposed in the form of lines in a direction that is substantially parallel to the flow of the test sample through the assay device.

In conventional lateral flow sandwich devices, uncomplexed analyte would compete with the complexed analyte for the capture reagent located at the detection zone, causing a drop off in the indication of the presence of the analyte. In a graphical representation of signal strength versus time, this drop off resembles a hook, hence this phenomenon is known as the “hook effect”. Depositing the test sample downstream from the conjugate particles results in some analyte complexing with the capture reagent before contact with the conjugate particles. This generally results in all or substantially all of the capture sites of the reagent being occupied by analyte. The conjugate particles subsequently form the new ternary sandwich complex upon their arrival at the detection zone. This sequence helps eliminate the “hook effect” found in previous assays because the analyte binds to virtually all of the capture reagent, (provided that there is sufficient analyte) and an excess of detection probes ensures that virtually all capture reagent sites contain complexed analyte.

Referring again to FIG. 1, the porous membrane 22 may also contain a control zone 32 positioned downstream from the detection zone 30. The control zone 32 generally provides a single distinct region (e.g., line, dot, etc.), although multiple regions are certainly contemplated by the present invention. For instance, in the illustrated embodiment, a single line is utilized. The control zone 32 may be disposed in a direction that is substantially perpendicular to the flow of the buffer and detection probes through the device 20. Likewise, in some embodiments, the zone 32 may be disposed in a direction that is substantially parallel to the flow through the device 20.

Regardless of its configuration, a second capture reagent may be immobilized on the porous membrane 22 within the control zone 32. The second capture reagent serves as a stationary binding site for any conjugate particles and/or analyte/conjugated particle complexes that do not bind to the first capture reagent at the detection zone 30. Because it is desired that the second capture reagent bind to both complexed and uncomplexed conjugate particles, the second capture reagent is normally different from the first capture reagent. In one embodiment, the second capture reagent is a biological capture reagent (e.g., antigens, haptens, protein A or G, neutravidin, avidin, streptavidin, primary or secondary antibodies (e.g., polyclonal, monoclonal, etc.), and complexes thereof) that is different than the first capture reagent. For example, the first capture reagent may be a monoclonal antibody (e.g., CRP Mab1), while the second capture reagent may be avidin (a highly cationic 66,000-dalton glycoprotein), streptavidin (a nonglycosylated 52,800-dalton protein), neutravidin (a deglysolated avidin derivative), and/or captavidin (a nitrated avidin derivative). In this embodiment, the second capture reagent may bind to biotin, which is biotinylated or contained on detection probes conjugated with a monoclonal antibody different than the monoclonal antibody of the first capture reagent (e.g., CRP Mab2).

In addition, it may also be desired to utilize various non-biological materials for the second capture reagent of the control zone 32. In many instances, such non-biological capture reagents may be particularly desired to better ensure that all of the remaining conjugated detection probes and/or analyte/conjugated probe complex. An example is a polyelectrolyte material. Fluorescence detection may be used to detect the presence of analyte in the detection and control zones and generally utilizes wavelength filtering to isolate the emission photons from the excitation photons, and a detector that registers emission photons and produces a recordable output, usually as an electrical signal or a photographic image. Examples of the types of detectors include spectrofluorometers and microplate readers; fluorescence microscopes; fluorescence scanners; and flow cytometers. One suitable fluorescence detector for use with the present invention is a FluoroLog III Spectrofluorometer, which is sold by SPEX Industries, Inc. of Edison, N.J.

If desired, a technique known as “time-resolved fluorescence detection” may also be utilized in the present invention. Time-resolved fluorescence detection is designed to reduce background signals from the emission source or from scattering processes (resulting from scattering of the excitation radiation) by taking advantage of the fluorescence characteristics of certain fluorescent materials, such as lanthanide chelates of europium (Eu (III)) and terbium (Tb (III)). Such chelates may exhibit strongly red-shifted, narrow-band, long-lived emission after excitation of the chelate at substantially shorter wavelengths. Typically, the chelate possesses a strong ultraviolet absorption band due to a chromophore located close to the lanthanide in the molecule. Subsequent to light absorption by the chromophore, the excitation energy may be transferred from the excited chromophore to the lanthanide. This is followed by a fluorescence emission characteristic of the lanthanide. The use of pulsed excitation and time-gated detection, combined with narrow-band emission filters, allows for specific detection of the fluorescence from the lanthanide chelate only, rejecting emission from other species present in the sample that are typically shorter-lived or have shorter wavelength emission.

### EXAMPLE

This idea was tested with the following experimental setup:

Goat anti-mouse antibody (“GAM”) was diluted in phosphate-buffered saline (PBS) (pH of 7.2) to 0.1 mg/ml, and striped onto Millipore nitrocellulose HF120 membranes using a Kinematic 1600 coating machine at a dispense rate of 1 ul/cm and a bed speed of 5 cm/s. Scipac C-reactive protein (CRP) (Sittingbourne, Kent, UK) was diluted in water to give a final concentration of 2.6 mg/ml and was striped below the GAM test line at a dispense rate of 1 ul/cm. The cards were left to dry at 37° C. for 1 hour.

VF2 (from Whatman Corp., Clifton, N.J.) and GF33 (glass fiber) conjugate pad material (from Millipore Corp., Billerica, Mass.) was cut to 30 mm by 34 mm bands using a hand operated guillotine cutter. The monoclonal antibody for the conjugate was Mab1 (catalog number 10-C07, from Fitzgerald Industries International, Inc. of Concord, Mass. 01742-3049 USA). This CRP antibody was conjugated to 20 nm diameter gold particles. The resulting conjugate was mixed 1:1 with goat anti-rabbit (GAR) conjugate gold particles (40 nm diameter). The anti-CRP stock conjugate is at an optical density (OD) of 32, so when it is mixed with GAR in a 1:1 ratio it reduces it to OD 16. This was further diluted in 2 mM Borax (pH 7.2) and 50% sucrose (final 10% sucrose) to give a final OD of 10. Borax is one of a few buffer types that are effective and sucrose or other hydrophilic materials aid in re-suspension of the dried particles due to their high solubility in water).

Conjugate was sprayed at 5 ul/cm, 5 cm/s using the Kinematic 1600 coating machine onto the VF2 bands, 10 mm away from the edge of the band and 3 mm away from the opposite edge for control bands. These were left to dry overnight at less than 20 percent relative humidity and room temperature. The conjugate bands and wicking material (CF6 from Whatman) were cut to 20 mm wide bands and were laminated onto the striped HF120 membrane. The laminated bands were then cut to 4 mm wide strips using the Kinematic 2360 to make dipsticks.

Either EDTA-treated whole blood or calibrated sera (Kamaya standards, Seattle, Wash.) were used in these experiments. Scipac CRP was spiked into the whole blood to give final concentrations of 2.5, 10, 20, 40, 80 and 160 ug/ml. Kamaya standard sera were used for GF33 experiments. Whole blood in an amount of 1 ul was added approximately 13 mm from the bottom of the VF2 pad in a line using a positive displacement pipette for control test strips, while 1 ul of blood was added immediately after the sprayed conjugate band (˜13 mm from end of band) for the inventive assay. PBS with 2% TWEEN® 20 surfactant (from Sigma-Aldrich Chemical Co.) was then added to a buffer reservoir at the end opposite the wicking strip and the lid of the housing was clamped in place. The test strips were left to run for 30 minutes before reading the results visually.

The inventive assay resulted in much stronger signals at the test line. For example for this specific case of an “inhibition/overflow assay”, the GAM line, which acts as the signal line since its intensity increases with increasing levels of analyte (CRP in this case), had much stronger signals when this method was used.

In addition to applying the blood sample at 13 mm from the base of the test strip as above, other positions for the blood sample were evaluated. These ranged from 5 mm to 29 mm, as measured from the base of the test strip. All of these positions can affect the outcome of the test signal (e.g., line intensity or quality). Specifically, it was found that the closer a sample (e.g., blood) is applied to the base, the clearer the background becomes thereby improving the signal at the test line. This signal improvement was either in the form of a stronger, more intense line; better line quality; or lower background signal on the other areas of the test strip.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A lateral flow assay device for detecting the presence or quantity of an analyte residing in a test sample, said lateral flow assay device comprising a porous membrane, said porous membrane being in communication with a conjugate pad and a wicking pad, said porous membrane defining:
a detection zone within which is immobilized a first capture reagent, said first capture reagent being configured to bind to at least a portion of said analyte and analyte-conjugate complexes to generate a detection signal having an intensity; and,
said conjugate pad located upstream from said detection zone, said conjugate pad having detection particles with specific binding members for the analyte and;
a buffer release zone located upstream of said conjugate pad and providing for buffer addition to said device, said buffer serving to move said detection probes to said detection zone, and;
said sample being deposited between said conjugate pad and said detection zone.

2. A lateral flow assay device as defined in claim 1, wherein said conjugated detection particles comprise a substance selected from the group consisting of chromogens, catalysts, luminescent compounds, radioactive compounds, visual labels, liposomes, and combinations thereof.

3. A lateral flow assay device as defined in claim 1, wherein said conjugated detection particles comprise a luminescent compound.

4. A lateral flow assay device as defined in claim 1, wherein said conjugated detection particles comprise a visual label.

5. A lateral flow assay device as defined in claim 1, wherein said specific binding member is selected from the group consisting of antigens, haptens, aptamers, primary or secondary antibodies, biotin, and combinations thereof.

6. A lateral flow assay device as defined in claim 1, wherein said first capture reagent is selected from the group consisting of antigens, haptens, protein A or G, neutravidin, avidin, streptavidin, captavidin, primary or secondary antibodies, and complexes thereof.

7. A lateral flow assay device as defined in claim 1, wherein said second capture reagent is selected from the group consisting of antigens, haptens, protein A or G, neutravidin, avidin, streptavidin, captavidin, primary or secondary antibodies, and complexes thereof.

8. A lateral flow assay device as defined in claim 1, wherein said analyte is a large pathogen selected from the group consisting of Salmonella species, Neisseria meningitides groups, Streptococcus pneumoniae, Candida albicans, Candida tropicalis aspergillua, haemophilus influenza, HIV, Trichomonas and Plasmodium.

9. A lateral flow assay device as defined in claim 1, wherein said analyte is selected from the group consisting of toxins, organic compounds, proteins, peptide, microorganisms, amino acids, nucleic acids, hormones, steroids, vitamins, drugs, drug intermediaries or byproducts, bacteria, virus particles and metabolites of or antibodies to any of the above substances.

10. A lateral flow assay device as defined in claim 1, wherein said porous membrane, conjugate pad and wicking pad are made from a single material.

11. A method for detecting the presence or quantity of an analyte residing in a test sample, said method comprising:
i) providing a lateral flow assay device comprising a porous membrane, in liquid communication with a conjugate pad and a wicking pad, said conjugate pad having detection particles conjugated with a specific binding member for the analyte, said porous membrane defining a detection zone in which a first capture reagent is immobilized, and a control zone within which a second capture reagent is immobilized, wherein said control zone is located downstream from said detection zone, said conjugate pad is located upstream of said porous membrane and said buffer release zone is upstream of said conjugate pad;
ii) contacting said test sample containing the analyte between said conjugate pad and said detection zone;
iii) releasing a buffer at said buffer release zone so that said buffer will carry said detection particles to said detection and control zones;
iv) detecting a detection signal.

12. A method as defined in claim 11, wherein said conjugated detection particles comprise a substance selected from the group consisting of chromogens, catalysts, luminescent compounds, radioactive compounds, visual labels, liposomes, and combinations thereof.

13. A method as defined in claim 11, wherein said conjugated detection particles comprise a visual label.

14. A method as defined in claim 11, wherein said specific binding member is selected from the group consisting of antigens, haptens, aptamers, primary or secondary antibodies, biotin, and combinations thereof.

15. A method as defined in claim 11, wherein said first capture reagent is selected from the group consisting of antigens, haptens, protein A or G, neutravidin, avidin, streptavidin, captavidin, primary or secondary antibodies, and complexes thereof.

16. A method as defined in claim 11, wherein said second capture reagent is selected from the group consisting of antigens, haptens, protein A or G, neutravidin, avidin, streptavidin, captavidin, primary or secondary antibodies, and complexes thereof.

17. A method as defined in claim 11, wherein said second capture reagent comprises a polyelectrolyte.

18. A method as defined in claim 11, wherein said analyte is a large pathogen selected from the group consisting of Salmonella species, Neisseria meningitides groups, Streptococcus pneumoniae, Candida albicans, Candida tropicalis, aspergillua, haemophilus influenza, HIV, Trichomonas and Plasmodium.

19. A method as defined in claim 11, wherein said analyte is selected from the group consisting of toxins, organic compounds, proteins, peptides, microorganisms, amino acids, nucleic acids, hormones, steroids, vitamins, drugs, drug intermediaries or byproducts, bacteria, virus particles and metabolites of or antibodies to any of the above substances.

20. A lateral flow assay device for detecting the presence of an analyte residing in a test sample, wherein detection particles, initially located on a conjugate pad, are moved to a pathogen located in a detection zone having a capture reagent.
